(19) United States
(12) Patent Application Publication
(10) Pub. No.: US 2025/0143937 A1
(43) Pub. Date: May 8, 2025

(54) REDUCED BASIS WEIGHT ACQUISITION DISTRIBUTION LAYER

(71) Applicant: Berry Global, Inc., Evansville, IN (US)

(72) Inventors: Sylvie Lesne-Olivier, Neuville en Ferrain (FR); Robert Garcia, Alcanar (ES)

(21) Appl. No.: 18/926,373

(22) Filed: Oct. 25, 2024

Related U.S. Application Data
(60) Provisional application No. 63/547,832, filed on Nov. 8, 2023.
(60) Provisional application No. 63/656,830, filed on Jun. 6, 2024.

(51) Int. Cl. A61F 13/531; A61F 13/15; A61F 13/53

(52) U.S. Cl. CPC A61F 13/531; A61F 13/15739; A61F 2013/15406; A61F 2013/530299; A61F 2013/530868; A61F 2013/530897; A61F 2013/5315

(57) ABSTRACT

Absorbent articles, such as channeled core diapers, including (i) a topsheet; (ii) an acquisition distribution layer (ADL) comprising a first spunbond nonwoven comprising a first plurality of hydrophilic polyolefin fibers, wherein the ADL has a basis weight from about 5 to about 20 grams-per-meter-squared (gsm), such as at least about any of the following: 5, 6, 7, 8, 9, 10, 11, and 12 gsm, and/or at most about any of the following: 20, 19, 18, 17, 16, 15, 14, 13, and 12 gsm; and (iii) an absorbent core including (a) a core warp layer and (b) one or more channels defining one or more troughs, wherein the ADL is located directly or indirectly between the topsheet and the core wrap layer of the absorbent core.

### CROSS-REFERENCE TO RELATED APPLICATIONS

This application claims priority under 35 U.S.C. § 119 to U.S. Provisional Applications No. 63/547,832 filed Nov. 8, 2023 and 63/656,830 filed Jun. 6, 2024, which are each expressly incorporated by reference herein in their entirety.

### TECHNICAL FIELD

Embodiments of the presently-disclosed invention relate generally to absorbent articles (e.g., channeled core diapers) including an acquisition distribution layer (ADL) comprising a spunbond nonwoven having a reduced basis weight while providing desirable Rewet and Liquid Strike Through Time (LSTT) values.

### BACKGROUND

A variety of absorbent articles that are adapted to absorb body fluids are well known. Examples of absorbent articles include diapers, feminine hygiene products, and incontinence guards. These products typically include a top sheet facing the body of the user, a back sheet facing the garment of a user, and an absorbent layer sandwiched between the inner, top sheet and outer, back sheet.

One problem associated with known absorbent articles is waste product leakage, which may be reduced by increasing the rate that liquid enters into the absorbent core. For instance, an absorbent article that enables liquid to rapidly penetrate the topsheet and be contained, such as in the absorbent core, will experience less leakage than an absorbent article wherein liquid is able to run across the topsheet before penetrating into the absorbent core. Consequently, run-off reduction reduces the amount of leakage associated with an absorbent article.

Another problem associated with absorbent articles is failure to maintain dryness of the skin contacting surface (e.g., the topsheet) of the absorbent article. Generally, the drier the skin contacting surface (e.g., the topsheet), the more comfortable the absorbent article is for the user. Traditionally, therefore, increased physical separation between the absorbent core and the skin contacting surface has been desired and pursued, such as by the use of lofty and/or high basis weight intermediate layers like ADLs that may provide a large void volume therein.

There is still a need in the art, however, for absorbent articles that provide desirably fast LSTT with reduced Rewet values, particularly with the utilization of reduced materials (e.g., reduced basis weight of the absorbent article, the ADL, and/or the topsheet.

### SUMMARY OF INVENTION

One or more embodiments of the invention may address one or more of the aforementioned problems. Certain embodiments according to the invention provide an absorbent article, comprising the following: (i) a topsheet; (ii) an acquisition distribution layer (ADL) comprising a first spunbond nonwoven fabric comprising a first plurality of hydrophilic polyolefin fibers, wherein the ADL has a basis weight from about 5 to about 20 grams-per-meter-squared (gsm), such as at least about any of the following: 5, 6, 7, 8, 9, 10, 11, and 12 gsm, and/or at most about any of the following: 20, 19, 18, 17, 16, 15, 14, 13, and 12 gsm; and (iii) an absorbent core, which may be at least partially (e.g., completely) enclosed by a core-wrap, wherein the ADL is located directly between the topsheet and the absorbent core or indirectly between the topsheet and the absorbent core (e.g., a core-wrap material may be located between the ADL and the absorbent core).

Certain embodiments according to the invention provide an absorbent article, comprising the following: (i) a topsheet; (ii) an acquisition distribution layer (ADL) comprising a first nonwoven fabric comprising a Sₓ-M_{y}-S_{z} construction, wherein (a) ‘x’ has a value from 1-10, (b) ‘y’ has a value from 1-10, and (c) ‘z’ has a value from 1-10; in which the first nonwoven fabric comprises a first plurality of hydrophilic polyolefin fibers, wherein the ADL has a basis weight from about 5 to about 18 grams-per-meter-squared (gsm), such as at least about any of the following: 5, 6, 7, 8, 9, 10, 11, and 12 gsm, and/or at most about any of the following: 20, 19, 18, 17, 16, 15, 14, 13, and 12 gsm; and (iii) an absorbent core including (a) a core warp layer and (b) optionally one or more channels defining one or more troughs, wherein the ADL is located directly or indirectly between the topsheet and the core wrap layer of the absorbent core.

Certain embodiments according to the invention provide an absorbent article, comprising the following: (i) a topsheet; (ii) an acquisition distribution layer (ADL) comprising a first spunbond nonwoven fabric or first spunmelt nonwoven fabric comprising a first plurality of hydrophilic polyolefin fibers, wherein the ADL has a basis weight from about 5 to about 20 grams-per-meter-squared (gsm), such as at least about any of the following: 5, 6, 7, 8, 9, 10, 11, and 12 gsm, and/or at most about any of the following: 20, 19, 18, 17, 16, 15, 14, 13, and 12 gsm; and (iii) an absorbent core including (a) a core warp layer and (b) one or more channels defining one or more troughs, wherein the ADL is located directly or indirectly between the topsheet and the core wrap layer of the absorbent core.

In another aspect, the present invention provides a method of making an absorbent article comprising the following steps: (i) providing or forming a topsheet; (ii) providing or forming an acquisition distribution layer (ADL) comprising a first spunbond nonwoven fabric comprising a first plurality of hydrophilic polyolefin fibers (e.g., polyolefin spunbond fibers having a hydrophilic treatment/additive thereon), wherein the ADL has a basis weight from about 5 to about 20 grams-per-meter-squared (gsm), such as at least about any of the following: 5, 6, 7, 8, 9, 10, 11, and 12 gsm, and/or at most about any of the following: 20, 19, 18, 17, 16, 15, 14, 13, and 12 gsm; (iii) providing or forming an absorbent core, which may be at least partially (e.g., completely) enclosed by a core-wrap; (iv) directly or indirectly bonding the topsheet to a first surface of the ADL; (v) directly or indirectly bonding the absorbent core to a second surface of the ADL, wherein the ADL is located directly between the topsheet and the absorbent core or indirectly between the topsheet and the absorbent core (e.g., a core-wrap material may be located between the ADL and the absorbent core) to provide an absorbent article, such as those described and disclosed herein.

### BRIEF DESCRIPTION OF THE DRAWING(S)

The invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout, and wherein:

FIG. 1 illustrates the relative positioning of a topsheet, an ADL, and an absorbent core in accordance with certain embodiments of the invention;

FIGS. 2A-2C illustrate example absorbent cores including at least on channel formed therein, in accordance with certain embodiments of the invention, while FIG. 2D illustrates a cross-sectional view of an absorbent article illustrating an absorbent core including channels in accordance with certain embodiments of the invention;

FIG. 3 shows test data for Rewet values associated with a variety of samples;

FIG. 4 shows test data for LSTT values associated with a variety of samples; and

FIG. 5 is a plot of the Rewet values from FIG. 3 vs. the LSTT values from FIG. 4.

### DETAILED DESCRIPTION

The invention now will be described more fully hereinafter. Indeed, this invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

Certain embodiments of the invention may be directed to absorbent articles including an ADL having a reduced basis weight and/or thickness, while simultaneously exhibiting a combination of both desirably low Rewet values and low LSTT. In this regard, absorbent articles, in accordance with certain embodiments of the invention, may provide the same as or superior liquid handling properties for absorbent articles despite utilizing a significantly reduced grammage of material (e.g., reduced ADL and/or topsheet basis weights). In this regard, a greater number of absorbent articles may be produced from a given amount of nonwoven material. Moreover, the thinner and/or lighter nature of the absorbent article may provide increased comfort to a user. The absorbent articles may include a spin-finish applied to the ADL, the topsheet, and/or a core-wrap of an absorbent core of the absorbent article.

The term “machine direction” or “MD”, as used herein, comprises the direction in which the fabric produced or conveyed. The term “cross-direction” or “CD”, as used herein, comprises the direction of the fabric substantially perpendicular to the MD.

The terms “nonwoven” and “nonwoven web”, as used herein, may comprise a web having a structure of individual fibers, fibers, and/or filaments that are interlaid but not in an identifiable repeating manner as in a knitted or woven fabric. Nonwoven fabrics or webs, according to certain embodiments of the invention, may be formed by any process conventionally known in the art such as, for example, meltblowing processes, spunbonding processes, needle-punching, hydroentangling, air-laid, and bonded carded web processes. A “nonwoven web”, as used herein, may comprise a plurality of individual fibers that have not been subjected to a consolidating process. In certain instances, the “nonwoven web” may comprises a plurality of layers, such as one or more spunbond layers and/or one or more meltblown layers. For instance, a “nonwoven web” may comprises a spunbond-meltblown-spunbond structure.

The terms “fabric” and “nonwoven fabric”, as used herein, may comprise a web of fibers in which a plurality of the fibers are mechanically entangled or interconnected, fused together, and/or chemically bonded together. For example, a nonwoven web of individually laid fibers may be subjected to a bonding or consolidation process to bond at least a portion of the individually fibers together to form a coherent (e.g., united) web of interconnected fibers.

The term “consolidated” and “consolidation”, as used herein, may comprise the bringing together of at least a portion of the fibers of a nonwoven web into closer proximity or attachment there-between (e.g., thermally fused together, chemically bonded together, and/or mechanically entangled together) to form a bonding site, or bonding sites, which function to increase the resistance to external forces (e.g., abrasion and tensile forces), as compared to the unconsolidated web. The bonding site or bonding sites, for example, may comprise a discrete or localized region of the web material that has been softened or melted and optionally subsequently or simultaneously compressed to form a discrete or localized deformation in the web material. Furthermore, the term “consolidated” may comprise an entire nonwoven web that has been processed such that at least a portion of the fibers are brought into closer proximity or attachment there-between (e.g., thermally fused together, chemically bonded together, and/or mechanically entangled together), such as by thermal bonding or mechanical entanglement (e.g., hydroentanglement) as merely a few examples.

The term “hydrophilic”, as used herein, may refer to a material (e.g., fibers, nonwoven web and/or fabric, etc.) having a surface that tends to absorb water or be wetted by water. In this regard, a material or surface may be considered “hydrophilic” if water droplets deposited thereon spreads, wetting a large area of the surface, then the contact angle is less than 90° and that surface may be designated as being hydrophilic

As used herein, the term “fiber” may refer to a staple fiber, a meltblown fiber, and/or a continuous spunbond filament. In this regard, the use of the term “spunbond fiber” may be used interchangeably with “spunbond filament”. A fiber typically means an elongate particulate having an apparent length exceeding its apparent width, substantially exceeding according to certain embodiments of the invention.

As used herein, the term “continuous fibers” refers to fibers which are not cut from their original length prior to being formed into a nonwoven web or nonwoven fabric. Continuous fibers, such as spunbond fibers, may have average lengths ranging from greater than about 15 centimeters to more than one meter, and up to the length of the web or fabric being formed. For example, a continuous fiber, as used herein, may comprise a fiber in which the length of the fiber is at least 1,000 times larger than the average diameter of the fiber, such as the length of the fiber being at least about 5,000, 10,000, 50,000, or 100,000 times larger than the average diameter of the fiber.

The term “staple fiber”, as used herein, may comprise a cut fiber from a filament. In accordance with certain embodiments, any type of filament material may be used to form staple fibers. For example, staple fibers may be formed from regenerated cellulose, polymeric fibers, and/or elastomeric fibers. The average length of staple fibers may comprise, by way of example only, from about 2 centimeter to about 15 centimeter.

The term “meltblown”, as used herein, may comprise fibers formed by extruding a molten thermoplastic material through a plurality of fine die capillaries as molten threads or filaments into converging high velocity, usually hot, gas (e.g. air) streams which attenuate the filaments of molten thermoplastic material to reduce their diameter, which may be to microfiber diameter, according to certain embodiments of the invention. According to an embodiment of the invention, the die capillaries may be circular. Thereafter, the meltblown fibers are carried by the high velocity gas stream and are deposited on a collecting surface to form a web of randomly disbursed meltblown fibers. Meltblown fibers may comprise microfibers which may be continuous or discontinuous and are generally tacky when deposited onto a collecting surface. Meltblown fibers, however, are shorter in length than those of spunbond fibers.

In one aspect, the present invention provides an absorbent article, comprising the following: (i) a topsheet; (ii) an acquisition distribution layer (ADL) comprising a first spunbond nonwoven fabric comprising a first plurality of hydrophilic polyolefin fibers, wherein the ADL has a basis weight from about 5 to about 20 grams-per-meter-squared (gsm), such as at least about any of the following: 5, 6, 7, 8, 9, 10, 11, and 12 gsm, and/or at most about any of the following: 20, 19, 18, 17, 16, 15, 14, 13, and 12 gsm; and (iii) an absorbent core, which may be at least partially (e.g., completely) enclosed by a core-wrap, wherein the ADL is located directly between the topsheet and the absorbent core or indirectly (e.g., a core-wrap material may be located between the ADL and the absorbent core) between the topsheet and the absorbent core. In accordance with certain embodiments of the invention, the absorbent article may comprise a liquid impermeable back sheet, such as a film, in which the absorbent core may be located directly or indirectly between the back sheet and the ADL.

Certain embodiments according to the invention provide an absorbent article, comprising the following: (i) a topsheet; (ii) an acquisition distribution layer (ADL) comprising a first nonwoven fabric comprising a Sₓ-M_{y}-S_{z} construction, wherein (a) ‘x’ represents a number of layers and has a value from 1-10, (b) ‘y’ represents a number of layers and has a value from 1-10, and (c) ‘z’ represents a number of layers and has a value from 1-10; in which the first nonwoven fabric comprises a first plurality of hydrophilic polyolefin fibers, wherein the ADL has a basis weight from about 5 to about 18 grams-per-meter-squared (gsm), such as at least about any of the following: 5, 6, 7, 8, 9, 10, 11, and 12 gsm, and/or at most about any of the following: 20, 19, 18, 17, 16, 15, 14, 13, and 12 gsm; and (iii) an absorbent core including (a) a core warp layer and (b) optionally one or more channels defining one or more troughs, wherein the ADL is located directly or indirectly between the topsheet and the core wrap layer of the absorbent core. In accordance with certain embodiments of the invention, ‘x’ may have a value of 1, 2, 3, 4, 5, 6, 7, 8, 9, or 10; ‘y’ may have value of 1, 2, 3, 4, 5, 6, 7, 8, 9, or 10; and ‘z’ may have a value of 1, 2, 3, 4, 5, 6, 7, 8, 9, or 10. Additionally or alternatively, the first plurality of hydrophilic polyolefin fibers may comprise spunbond fibers/filaments, meltblown fibers, or both. Additionally or alternatively, the meltblown layer(s) may account for about 1 to about 20% by weight of a total fiber content of the first nonwoven fabric, such as at least about any of the following: 1, 2, 3, 4, 5, 6, 8, and 10% by weight of a total fiber content of the first nonwoven fabric, and/or at most about any of the following: 20, 18, 16, 14, 12, and 10% by weight of a total fiber content of the first nonwoven fabric.

Certain embodiments according to the invention provide an absorbent article, comprising the following: (i) a topsheet; (ii) an acquisition distribution layer (ADL) comprising a first spunbond nonwoven fabric or first spunmelt nonwoven fabric (e.g., a SMS nonwoven fabric) comprising a first plurality of hydrophilic polyolefin fibers, wherein the ADL has a basis weight from about 5 to about 20 grams-per-meter-squared (gsm), such as at least about any of the following: 5, 6, 7, 8, 9, 10, 11, and 12 gsm, and/or at most about any of the following: 20, 19, 18, 17, 16, 15, 14, 13, and 12 gsm; and (iii) an absorbent core including (a) a core warp layer and (b) one or more channels defining one or more troughs, wherein the ADL is located directly or indirectly between the topsheet and the core wrap layer of the absorbent core

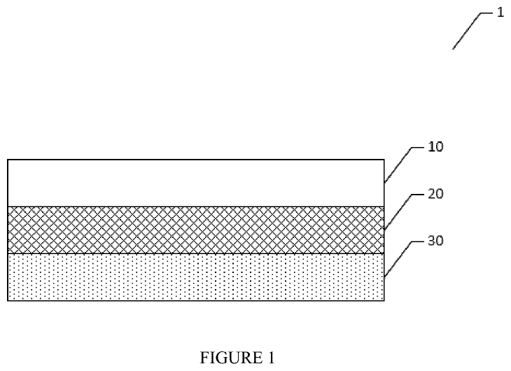

FIG. 1, for instance, illustrates a portion of an absorbent article 1 showing the relative positioning of a topsheet 10, an ADL 20, and an absorbent core 30 in accordance with certain embodiments of the invention. In this regard, the topsheet 10 may be located adjacent and/or in contact with the ADL 20. Similarly the ADL 20 may be located adjacent and/or in contact with the absorbent core 30 (e.g., the core-wrap of the absorbent core). In this regard, the individual layers may be bonded together via an adhesive and/or thermal bonds.

In accordance with certain embodiments of the invention, the first plurality of hydrophilic polyolefin fibers comprise a polymer component and optionally an additive component, such as provided in the polymer melt used for forming the first plurality of hydrophilic polyolefin fibers. In this regard, the polymer component may comprise a polypropylene, a polypropylene copolymer, a polyethylene, a polyethylene copolymer, or blends thereof. The additive component may include, for example, processing aids, fillers, and/or stabilizers.

In accordance with certain embodiments of the invention, the first plurality of hydrophilic polyolefin fibers comprise a hydrophilic spin-finish coated or otherwise disposed thereon. The spin-finish, for example, may be anionic, cationic, or non-ionic in nature. The spin-finish, for example, may include one or more anionic agents, one or more non-ionic agents, and/or one or more cationic agents (e.g., blends of cationic and non-ionic agents or blends of anionic and non-ionic agents, etc.) Additionally or alternatively, the spin-finish may be silicone-free or comprise a silicon-containing chemistry. One example of a suitable spin-finish is SILASTOL® 163 from, Schill+Seilacher GmbH, (Germany). Alternatively, the spin-finish may comprise a nonionic modified siloxane additive, such as a nonionic modified polyorganosiloxane. In certain embodiments, for example, the nonionic modified siloxane additive may comprise a polyoxyalkyl-modified polydimethyl siloxane. In some embodiments, for instance, the nonionic modified siloxane additive may comprise a polyoxyalkyl (C2-C8) polydimethyl siloxane. In such embodiments, for example, the nonionic modified siloxane additive may comprise a polyoxyalkyl (C2-C4) polydimethyl siloxane (e.g., SILASTOL® 186 F from Schill+Seilacher GmbH, Germany). Additionally or alternatively, the first plurality of hydrophilic polyolefin fibers may be formed from a melt additive to the polymeric composition forming the first plurality of fibers, in which the melt additive comprises a hydrophilic additive compound(s) such as (but not limited to) any of those noted above.

In accordance with certain embodiments of the invention, the ADL may comprise from about 0.3 to about 3% by weight (e.g., on a dry basis) of the spin-finish, such as at least about any of the following: 0.3, 0.5, 0.7, 0.8, and 1% by weight (e.g., on a dry basis), and/or at most about any of the following: 3, 2.8, 2.5, 2.2, 2, 1.8. 1.5, 1.2, and 1% by weight (e.g., on a dry basis).

The topsheet, in accordance with certain embodiments of the invention, may comprise a second spunbond nonwoven fabric comprising a second plurality of hydrophilic polyolefin fibers, such as those described and disclosed herein (e.g., polyolefin spunbond fibers rendered hydrophilic via treatment with a hydrophilic spin-finish). Additionally or alternatively, the topsheet may have a basis weight from about 5 to about 15 grams-per-meter-squared (gsm), such as at least about any of the following: 5, 6, 7, 8, 9, and 10 gsm, and/or at most about any of the following: 15, 14, 13, 12, 11, and 10 gsm.

The second plurality of hydrophilic polyolefin fibers, for example, may comprise a hydrophilic spin-finish coated or otherwise disposed thereon. The spin-finish, for example, may be anionic, cationic, or non-ionic in nature. The spin-finish, for example, may include one or more anionic agents, one or more non-ionic agents, and/or one or more cationic agents (e.g., blends of cationic and non-ionic agents or blends of anionic and non-ionic agents, etc.) Additionally or alternatively, the spin-finish may be silicone-free or comprise a silicon-containing chemistry. In accordance with certain embodiments of the invention, the second plurality of hydrophilic polyolefin fibers comprise a polymer component and optionally an additive component, such as provided in the polymer melt used for forming the second plurality of hydrophilic polyolefin fibers. In this regard, the polymer component may comprise a polypropylene, a polypropylene copolymer, a polyethylene, a polyethylene copolymer, or blends thereof. The additive component may include, for example, processing aids, fillers, and/or stabilizers. One example of a suitable spin-finish is SILASTOL® 163 from, Schill+Seilacher (Germany). Alternatively, the spin-finish may comprise a nonionic modified siloxane additive, such as a nonionic modified polyorganosiloxane. In certain embodiments, for example, the nonionic modified siloxane additive may comprise a polyoxyalkyl-modified polydimethyl siloxane. In some embodiments, for instance, the nonionic modified siloxane additive may comprise a polyoxyalkyl (C2-C8) polydimethyl siloxane. In such embodiments, for example, the nonionic modified siloxane additive may comprise a polyoxyalkyl (C2-C4) polydimethyl siloxane (e.g., SILASTOL® 186 F from Schill+Seilacher GmbH, Germany).

In accordance with certain embodiments of the invention, the topsheet may comprise from about 0.3 to about 3% by weight (e.g., on a dry basis) of the spin-finish, such as at least about any of the following: 0.3, 0.5, 0.7, 0.8, and 1% by weight (e.g., on a dry basis), and/or at most about any of the following: 3, 2.8, 2.5, 2.2, 2, 1.8. 1.5, 1.2, and 1% by weight (e.g., on a dry basis).

In accordance with certain embodiments of the invention, the topsheet and the ADL may utilize the same spin-finish. Additionally or alternatively, a weight percentage of the spin-finish of the ADL is larger than a weight percentage of the spin-finish of the topsheet. For example, the absorbent article may comprise a first ratio between the weight percentage of the spin-finish of the ADL to the weight percentage of the spin-finish of the topsheet from about 1.1:1 to about 3:1, such as at least about any of the following: 1.1:1, 1.4:1, 1.6:1, 1.8:1, and 2:1, and/or at most about any of the following: 3:1, 2.8:1, 2.6:1, 2.4:1, 2.2:1, and 2:1. Additionally or alternatively, a total weight amount of the spin-finish on a dry basis of the ADL is larger than a total weight amount of the spin-finish on a dry basis of the topsheet. For example, the absorbent article may comprise a second ratio between the total weight amount of the spin-finish on a dry basis of the ADL to the total weight amount of the spin-finish on a dry basis of the topsheet comprises from about 1.1:1 to about 3:1, such as at least about any of the following: 1.1:1, 1.4:1, 1.6:1, 1.8:1, and 2:1, and/or at most about any of the following: 3:1, 2.8:1, 2.6:1, 2.4:1, 2.2:1, and 2:1.

Alternatively, in accordance with certain embodiments of the invention, the topsheet and the ADL may utilize the same spin-finish, in which a weight percentage of the spin-finish of the ADL is smaller than a weight percentage of the spin-finish of the topsheet. For example, the absorbent article may comprise an alternative first ratio between the weight percentage of the spin-finish of the topsheet to the weight percentage of the spin-finish of the ADL from about 1.1:1 to about 3:1, such as at least about any of the following: 1.1:1, 1.4:1, 1.6:1, 1.8:1, and 2:1, and/or at most about any of the following: 3:1, 2.8:1, 2.6:1, 2.4:1, 2.2:1, and 2:1. Additionally or alternatively, a total weight amount of the spin-finish on a dry basis of the ADL may be smaller than a total weight amount of the spin-finish on a dry basis of the topsheet. For example, the absorbent article may comprise an alternative second ratio between the total weight amount of the spin-finish on a dry basis of the topsheet to the total weight amount of the spin-finish on a dry basis of the ADL comprises from about 1.1:1 to about 3:1, such as at least about any of the following: 1.1:1, 1.4:1, 1.6:1, 1.8:1, and 2:1, and/or at most about any of the following: 3:1, 2.8:1, 2.6:1, 2.4:1, 2.2:1, and 2:1

In accordance with certain embodiments of the invention, the ADL and topsheet define a combined basis weight from about 10 to about 35 gsm, such as at least about any of the following: 10, 12, 15, 18, 20, 22, and 24 gsm, and/or at most about any of the following: 35, 32, 30, 28, 26, and 24 gsm.

In accordance with certain embodiments of the invention, the absorbent core comprises one or more channels formed therein, such as by an embossing and/or a thermal bonding method, wherein the one or more channels define respective troughs that are open facing and proximate to the ADL and include respective bottom portions distally located from the ADL. The one or more channels may be configured to facilitate pooling and/or distribution of liquid across the one or more channels of the absorbent core. For example, the one or more channels may facilitate the distribution of liquid across both the MD and the CD of the absorbent core. Distribution of the liquid enables the use of a larger surface area of the absorbent core relative to the volume of liquid being handled. The one or more channels may have an average width at an open face portion, in accordance with certain embodiments of the invention, from about 1 to about 15 mm, such as at least about any of the following: 1, 3, 5, 6, and 8 mm, and/or at most about any of the following: 15, 12, 10, and 8 mm. Additionally or alternatively, the one or more channels may have an average depth measured from the respective open face portion to the respective bottom portion from about 0.5 to about 3 mm, such as at least about any of the following: 0.5, 0.8, 1, 1.2, and 1.5 mm, and/or at most about any of the following: 3, 2.5, 2, and 1.5 mm.

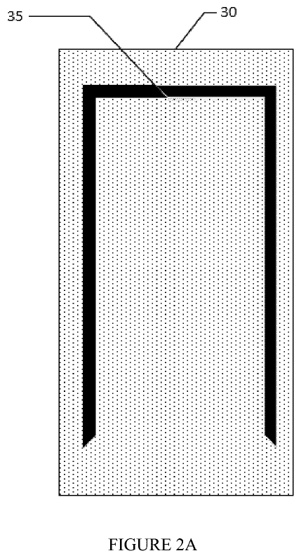

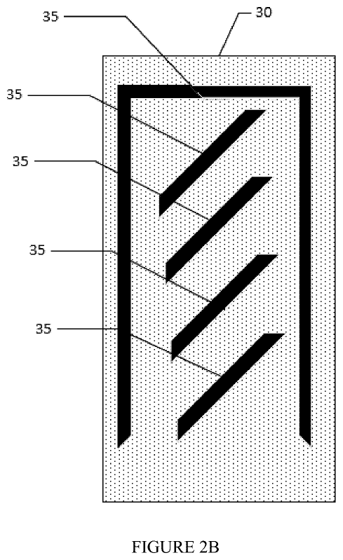

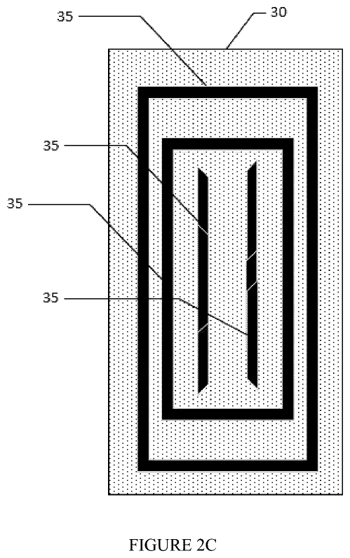

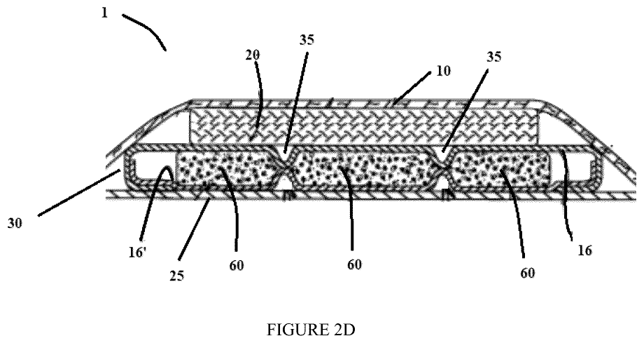

FIGS. 2A-2C, for instance, each illustrates an absorbent core 30 including at least one channel 35 formed therein, in accordance with certain embodiments of the invention. In this regard, the number and/or pattern of the one or more channels may not be particularly limited. Additionally, it should be appreciated that the one or more channels may be formed by the thermal bonding or embossing of one or more core-wrap layers (e.g., nonwoven fabric(s)) that at least partially encircle or define at least a portion of an outermost surface of the absorbent core. For example, the absorbent core may comprise at least one core-wrap (e.g., nonwoven fabric) at least partially (e.g., completely) enclosing one or more absorbent materials of the absorbent core, in which the absorbent materials may include, for example, super absorbent polymers, cellulosic fibers, and combinations thereof. In accordance with certain embodiments of the invention, the one or more core-wrap materials may include, for example, a top core-wrap layer adhered (e.g., thermally bonded, adhesively bonded, etc.) to a bottom core-wrap layer to form an interconnected absorbent material-free channel(s) 35 (generally in dry state). In this regard, the top core-wrap layer and the bottom core-wrap layer may be separate pieces of material (e.g., two separate materials bonded together) or a single unitary material bonded to itself). FIG. 2D, for example illustrates a cross-sectional view of an absorbent article 1 illustrating an absorbent core 30 including channels 35 in accordance with certain embodiments of the invention. The absorbent core 30, as noted above, may comprise an absorbent material and a core-wrap enclosing the absorbent material. The core wrap may comprise two separate materials, substrates, or nonwoven materials 16 and 16′ for the top side and bottom side of the absorbent core. In this regard, the absorbent core 30 may comprise one or more channels 35 formed therein. As shown in FIG. 2D the absorbent article 1 includes a top sheet 10, a liquid impermeable back sheet 25, an ADL 20, and an absorbent core 30, which includes a core-wrap formed from two separate core-wrap materials 16 and 16′ that encloses an absorbent material 60 of the absorbent core. As illustrated by FIG. 2D, the channels 35 may be defined by the interconnecting of the core-wrap materials 16 and 16′, in which the channels 35 are material free sections located underneath the ADL 20.

In accordance with certain embodiments of the invention, the absorbent core includes a core-wrap, which may at least partially encase or define at least an outermost portion of the absorbent core, comprising a nonwoven fabric that includes a hydrophilic spin-finish coated thereon. The spin-finish, for example, may include one or more anionic agents, one or more non-ionic agents, and/or one or more cationic agents (e.g., blends of cationic and non-ionic agents or blends of anionic and non-ionic agents, etc.) Additionally or alternatively, the spin-finish may be silicone-free or comprise a silicon-containing chemistry. Additionally or alternatively, the spin-finish may be silicone-free. One example of a suitable spin-finish is SILASTOL® 163 from, Schill+Seilacher GmbH (Germany). Alternatively, the spin-finish may comprise a nonionic modified siloxane additive, such as a nonionic modified polyorganosiloxane. In certain embodiments, for example, the nonionic modified siloxane additive may comprise a polyoxyalkyl-modified polydimethyl siloxane. In some embodiments, for instance, the nonionic modified siloxane additive may comprise a polyoxyalkyl (C2-C8) polydimethyl siloxane. In such embodiments, for example, the nonionic modified siloxane additive may comprise a polyoxyalkyl (C2-C4) polydimethyl siloxane (e.g., SILASTOL® 186 F from Schill+Seilacher GmbH, Germany).

In accordance with certain embodiments of the invention, the core-wrap may comprise from about 0.3 to about 3% by weight (e.g., on a dry basis) of the spin-finish, such as at least about any of the following: 0.3, 0.5, 0.7, 0.8, and 1% by weight (e.g., on a dry basis), and/or at most about any of the following: 3, 2.8, 2.5, 2.2, 2, 1.8. 1.5, 1.2, and 1% by weight (e.g., on a dry basis).

In accordance with certain embodiments of the invention, a total percentage of the spin-finish based on a combined basis weight of the ADL and the topsheet comprises from about 0.5% by weight (e.g., on a dry basis) to about 1% by weight (e.g., on a dry basis), such as at least about any of the following: 0.5, 0.6, 0.7, and 0.75% by weight (e.g., on a dry basis), and/or at most about any of the following: 1, 0.9, 0.8, and 0.75% by weight (e.g., on a dry basis).

In accordance with certain embodiments of the invention, the ADL is devoid of staple fibers, devoid of meltblown fibers, and/or devoid of cellulosic fibers. Additionally or alternatively, the ADL may include one or more apertures or alternatively be devoid of one or more apertures. Additionally or alternatively, the ADL is devoid of film layers. Additionally or alternatively, the topsheet is devoid of staple fibers, devoid of meltblown fibers, and/or devoid of cellulosic fibers. Additionally or alternatively, the absorbent article may be devoid of intervening nonwoven layer and/or film layers located between the topsheet and the ADL.

In accordance with certain embodiments of the invention, the absorbent article has a Rewet value from 0.2 to about 0.6 grams (g) according to Hytec Test TM04_9, such as at least about any of the following: 0.2, 0.25, 0.3, 0.35, and 0.4 g, and/or at most about any of the following: 0.6, 0.55, 0.5, 0.45, and 0.4 g. Additionally or alternatively, the absorbent article has a third ratio between the Rewet (g) to the basis weight of the ADL (gsm) of at most 0.04:1, such at most about any of the following: 0.04:1, 0.038:1, 0.035:1 (g/gsm), and/or at least about any of the following: 0.02:1, 0.025:1, 0.028:1, 0.030:1, 0.032:1, and 0.035:1 (g/gsm).

Additionally or alternatively, the absorbent article has a fourth ratio between the Rewet (g) to the combined basis weight of the ADL and topsheet (gsm) of at most 0.025:1, such at most about any of the following: 0.025:1, 0.022:1, 0.020:1 (g/gsm), and/or at least about any of the following: 0.01:1, 0.012:1, 0.015:1, 0.016:1, 0.018:1, and 0.020:1 (g/gsm).

In accordance with certain embodiments of the invention, the absorbent article has a Liquid Strike Through Time (LSTT) value from about 100 to about 250 seconds(s) according to Hytec Test TM04_9, such as at least about any of the following: 100, 110, 120, 130, 140, and 150 s, and/or at most about any of the following: 250, 240, 220, 200, 190, 180, 170, 160, and 150 s. In this regard, the Hytec Test TM04_9 subjects the sample (e.g., absorbent article) to a series of four (4) separate gushes of liquid and the time associated with penetration of the respective gush is recorded. The LSTT is the aggregate time from each of the four (4) gushes of liquid. For example, the first gush of liquid will have a ‘T1’, which is the time required for penetration by the sample, while subsequent gushes will have a ‘T2’, T3′, and ‘T4’ value. In this regard, the LSTT is the sum of ‘T1’, ‘T2’, ‘T3’, and ‘T4’. In accordance with certain embodiments of the invention, the ‘T1’ value for the absorbent article according to Hytec Test TM04_9 may be from about 3 to about 20 s, such as at least about any of the following: 3, 5, 6, 8, and 10 s, and/or at most about any of the following: 20, 18, 16, 15, 14, 12, and 10 s. Additionally or alternatively, the ‘T2’ value for the absorbent article according to Hytec Test TM04_9 may be from about 20 to about 60 s, such as at least about any of the following: 20, 25, 30, and 35 s, and/or at most about any of the following: 60, 55, 50, 45, 40, and 35 s. Additionally or alternatively, the ‘T3’ value for the absorbent article according to Hytec Test TM04_9 may be from about 50 to about 120 s, such as at least about any of the following: 50, 55, 60, 65, 70, 75, 80, 85, 90, 95, 100 s, and/or at most about any of the following: 120, 115, 110, 105, and 100 s. Additionally or alternatively, the ‘T4’ value for the absorbent article according to Hytec Test TM04_9 may be from about 75 to about 220 s, such as at least about any of the following: 75, 80, 85, 90, 95, 100 s, and/or at most about any of the following: 220, 200, 180, 150, 120, and 100 s. Additionally or alternatively, the absorbent article has a fifth ratio between the LSTT(s) to the basis weight of the ADL from about 8 to about 20 (s/gsm), such as at least about any of the following: 8, 10, 11, 12, 13, 14, and 15, and/or at most about any of the following: 20, 19, 18, 17, 16, and 15. Additionally or alternatively, the absorbent article has a sixth ratio between the LSTT(s) to the combined basis weight of the ADL and topsheet (gsm) from about 4 to about 10 (s/gsm), such as at least about any of the following: 4, 5, 6, and 7, and/or at most about any of the following: 10, 9, 8, 7, 6, and 5.

In accordance with certain embodiments of the invention, the absorbent article has a seventh ratio between LSTT(s) and Rewet (g) from about 350 to about 450 (s/g), such as at least about any of the following: 350, 360, 380, and 400, and/or at most about any of the following: 450, 420, and 400.

In another aspect, the present invention provides a method of making an absorbent article comprising the following steps: (i) providing or forming a topsheet; (ii) providing or forming an acquisition distribution layer (ADL) comprising a first spunbond nonwoven fabric comprising a first plurality of hydrophilic polyolefin fibers, wherein the ADL has a basis weight from about 5 to about 20 grams-per-meter-squared (gsm), such as at least about any of the following: 5, 6, 7, 8, 9, 10, 11, and 12 gsm, and/or at most about any of the following: 20, 19, 18, 17, 16, 15, 14, 13, and 12 gsm; (iii) providing or forming an absorbent core; (iv) directly or indirectly bonding the topsheet to a first surface of the ADL; (v) directly or indirectly bonding the absorbent core to a second surface of the ADL, wherein the ADL is located directly or indirectly between the topsheet and the absorbent core to provide an absorbent article, such as those described and disclosed herein.

In accordance with certain embodiments of the invention, the method may also comprise applying a spin-finish, such as those described and disclosed herein, onto the first plurality of hydrophilic polyolefin fibers. The topsheet, for example, may comprise a second plurality of hydrophilic polyolefin fibers (e.g., spunbond fibers) and the method may comprise applying a spin-finish, such as those described and disclosed herein, onto the first plurality of hydrophilic polyolefin fibers. The spin-finish may be applied in a variety of traditional methods, such spraying, nip-coating, etc.

### Examples

The present disclosure is further illustrated by then following examples, which in no way should be construed as being limiting. That is, the specific features described in the following examples are merely illustrative and not limiting.

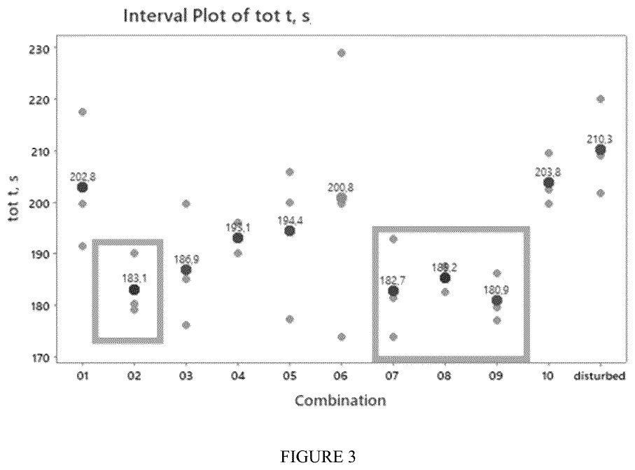

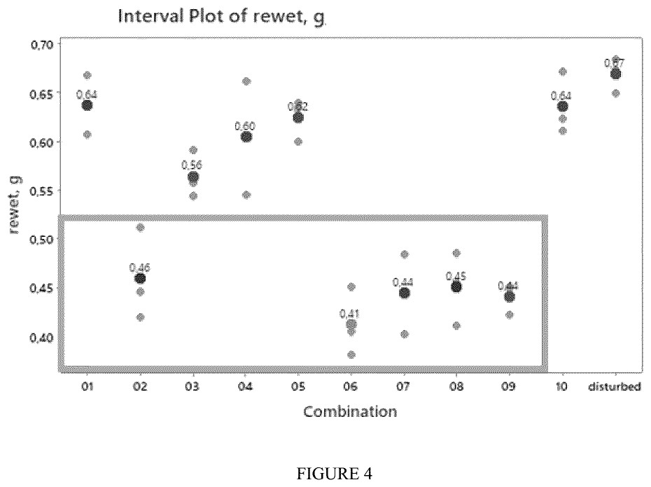

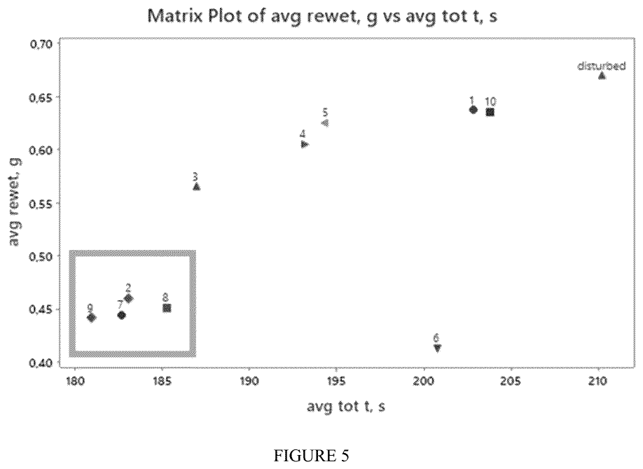

A commercially available diaper sold under the name “Lupilu Soft and Dry” from Lidl was compared to ten (10) different configurations, some of which exemplifying certain desirable features (e.g., reduced basis weight(s), improved lowered Rewet values and lowered LSTT values) in accordance with certain embodiments of the invention. Table 1 provides a summary of the Lupilu product and the ten (10) different configurations. The abbreviations used in Table 1 stand for the following: TS=topsheet; ADL=acquisition distribution layer; CW=core wrap; SB=Spunbond nonwoven; SMS=spunbond-meltblown-spunbond; and SF=spin-finish in weight percent, in which the spin-finish was SILASTOL® 163. Each of the samples (e.g., disturbed Lupilu product, and the 10 different configurations identified as 1-10 in Table 1) were tested for Rewet and LSTT according to Hytec Test TM04_9. The testing results for the Rewet values are provided in FIG. 3, while the LSTT values are provided in FIG. 4. FIG. 5 illustrates a plotting of the Rewet values vs. the LSTT values and highlights the significant simultaneous improvements in both Rewet and LSTT associated with certain embodiments of the invention (e.g., the samples associated with the data points enclosed in the box of FIG. 5).

TABLE 1

| Layer | Lupilu | 1 | 2 | 3 | 4 | 5 | 6 | 7 | 8 | 9 | 10 |
|---|---|---|---|---|---|---|---|---|---|---|---|
| TS | SB | SB | SB | SB | SB | SB | SB | SB | SB | SB | SB |
| | 16 gsm | 12 gsm | 12 gsm | 12 gsm | 13 gsm | 13 gsm | 12 gsm | 12 gsm | 12 gsm | 12 gsm | 12 gsm |
| | | 0.5% | 0.5% | 0.5% | 0.7% | 0.7% | 0.8% | 0.8% | 0.5% | 0.5% | 0.5% |
| | | SF | SF | SF | SF | SF | SF | SF | SF | SF | SF |
| ADL | SB | SB | SB | SB | SB | SB | SB | SB | SB | SB | SB |
| | 21 gsm | 16 gsm | 16 gsm | 29 gsm | 16 gsm | 29 gsm | 16 gsm | 16 gsm | 13 gsm | 12 gsm | 12 gsm |
| | | 0.5% | 0.7% | 0.5% | 0.5% | 0.5% | 0.5% | 0.7% | 0.7% | 0.8% | 0.5% |
| | | SF | SF | SF | SF | SF | SF | SF | SF | SF | SF |
| CW | SMS | SMS | SMS | SMS | SMS | SMS | SMS | SMS | SMS | SMS | SMS |
| | 12 gsm | 10 gsm | 10 gsm | 10 gsm | 10 gsm | 10 gsm | 10 gsm | 10 gsm | 10 gsm | 10 gsm | 10 gsm |

As illustrated by the data illustrated in FIGS. 3-5, the samples having configurations utilizing the combination of lower basis weights (e.g., for the ADL) with a somewhat higher spin-finish addition provided better performances. For example, Samples 2 and 7 utilized a higher spin-finish addition on the ADL, while Samples 8 and 9 utilized even lower basis weights for the ADL. In this regard, certain embodiments of the invention provide absorbent articles (e.g., diapers) that can provide the same or improved liquid handling properties, such as low Rewet and LSTT values, despite employing significantly lighter weight ADLs and/or topsheets. In this regard, the overall thickness and/or weight of individual absorbent articles may be significantly reduced.

These and other modifications and variations to the invention may be practiced by those of ordinary skill in the art without departing from the spirit and scope of the invention, which is more particularly set forth in the appended claims. In addition, it should be understood that aspects of the various embodiments may be interchanged in whole or in part. Furthermore, those of ordinary skill in the art will appreciate that the foregoing description is by way of example only, and it is not intended to limit the invention as further described in such appended claims. Therefore, the spirit and scope of the appended claims should not be limited to the exemplary description of the versions contained herein.

What is claimed is:

1. An absorbent article, comprising:
(i) a topsheet;
(ii) an acquisition distribution layer (ADL) comprising a first spunbond nonwoven fabric comprising a first plurality of hydrophilic polyolefin fibers, wherein the ADL has a basis weight from about 5 to about 20 grams-per-meter-squared (gsm);
(iii) an absorbent core, wherein the ADL is located directly or indirectly between the topsheet and the absorbent core.

2. The article of claim 1, wherein the first plurality of hydrophilic polyolefin fibers comprise a hydrophilic spin-finish coated thereon.

3. The article of claim 2, wherein the ADL comprises from about 0.3 to about 3% by weight of the hydrophilic spin-finish.

4. The article of claim 1, wherein the topsheet comprises a second spunbond nonwoven fabric comprising a second plurality of hydrophilic polyolefin fibers.

5. The article of claim 4, wherein the topsheet has a basis weight from about 5 to about 15 gsm.

6. The article of claim 5, wherein the second plurality of hydrophilic polyolefin fibers comprise a second hydrophilic spin-finish coated thereon.

7. The article of claim 6, wherein the topsheet comprises from about 0.3 to about 3% by weight of the second hydrophilic spin-finish.

8. The article of claim 6, wherein a first ratio between the weight percentage of the hydrophilic spin-finish of the ADL to the weight percentage of the second hydrophilic spin-finish of the topsheet comprises from about 1.1:1 to about 3:1.

9. The article of claim 1, wherein the ADL and topsheet define a combined basis weight from about 10 to about 35 gsm.

10. The article of claim 1, wherein the absorbent core comprises one or more channels formed therein, wherein the one or more channels define respective troughs that are open facing and proximate to the ADL and include respective bottom portions distally located relative to the ADL, the one or more channels being configured to facilitate pooling and/or distribution of liquid across the one or more channels of the absorbent core.

11. The article of claim 10, wherein the one or more channels has (i) an average width at an open face portion from about 1 to about 15 mm, (ii) an average depth measured from the respective open face portion to the respective bottom portion from about 0.5 to about 3 mm, or (iii) both (i) and (ii).

12. The article of claim 1, wherein the absorbent core includes a core-wrap comprising a nonwoven fabric that optionally includes a hydrophilic spin-finish coated thereon.

13. The article of claim 1, wherein the ADL is devoid of staple fibers, devoid of meltblown fibers, and/or devoid of cellulosic fibers.

14. The article of claim 1, wherein the ADL is devoid of apertures.

15. The article of claim 1, wherein the article has (i) a Rewet value from 0.2 to about 0.6 grams (g) according to Hytec Test TM04_9, (ii) a third ratio between the Rewet (g) to the basis weight of the ADL (gsm) of at most 0.04:1, such at most about any of the following: 0.04:1, 0.038:1, 0.035:1 (g/gsm), and/or at least about any of the following: 0.02:1, 0.025:1, 0.028:1, 0.030:1, 0.032:1, and 0.035:1 (g/gsm), (iii) a fourth ratio between the Rewet (g) to the combined basis weight of the ADL and topsheet (gsm) of at most 0.025:1, such at most about any of the following: 0.025:1, 0.022:1, 0.020:1 (g/gsm), and/or at least about any of the following: 0.01:1, 0.012:1, 0.015:1, 0.016:1, 0.018:1, and 0.020:1 (g/gsm), or (iv) any combination of (i), (ii), and/or (iii).

16. The article of claim 1, wherein the article has (i) a Liquid Strike Through Time (LSTT) value from about 100 to about 200 seconds(s) according to Hytec Test TM04_9, (ii) a fifth ratio between the LSTT(s) to the basis weight of the ADL from about 8 to about 20 (s/gsm), (iii) a sixth ratio between the LSTT(s) to the combined basis weight of the ADL and topsheet (gsm) from about 4 to about 10 (s/gsm), (iv) a sixth ratio between LSTT(s) and Rewet (g) from about 350 to about 450 (s/g), or (v) any combination of (i), (ii), (iii) and/or (iv).

17. An absorbent article, comprising:
(i) a topsheet;
(ii) an acquisition distribution layer (ADL) comprising a first nonwoven fabric comprising a Sₓ-M_{y}-S_{z} construction, wherein (a) ‘x’ has a value from 1-10, (b) ‘y’ has a value from 1-10, and (c) ‘z’ has a value from 1-10; the first nonwoven fabric comprises a first plurality of hydrophilic polyolefin fibers, wherein the ADL has a basis weight from about 5 to about 18 grams-per-meter-squared (gsm);
(iii) an absorbent core including (a) a core warp layer and (b) one or more channels defining one or more troughs, wherein the ADL is located directly or indirectly between the topsheet and the core wrap layer of the absorbent core.

18. The article of claim 17, wherein ‘x’ has a value of 1, 2, 3, 4, 5, 6, 7, 8, 9, or 10, ‘y’ has a value of 1, 2, 3, 4, 5, 6, 7, 8, 9, or 10, and ‘z’ has a value of 1, 2, 3, 4, 5, 6, 7, 8, 9, or 10.

19. An absorbent article, comprising:
(i) a topsheet;
(ii) an acquisition distribution layer (ADL) comprising a first spunbond nonwoven fabric or first spunmelt nonwoven fabric comprising a first plurality of hydrophilic polyolefin fibers, wherein the ADL has a basis weight from about 5 to about 20 grams-per-meter-squared (gsm);
(iii) an absorbent core including (a) a core warp layer and (b) one or more channels defining one or more troughs, wherein the ADL is located directly or indirectly between the topsheet and the core wrap layer of the absorbent core.

20. A method of making an absorbent article, comprising:
(i) providing or forming a topsheet;
(ii) providing or forming an acquisition distribution layer (ADL) comprising a first spunbond nonwoven comprising a first plurality of hydrophilic polyolefin fibers, wherein the ADL has a basis weight from about 5 to about 20 grams-per-meter-squared (gsm);
(iii) providing or forming an absorbent core;
(iv) directly or indirectly bonding the topsheet to a first surface of the ADL;
(v) directly or indirectly bonding the absorbent core to a second surface of the ADL, wherein the ADL is located directly or indirectly between the topsheet and the absorbent core.
